(19) United States
(12) Patent Application Publication
(10) Pub. No.: US 2018/0224390 A1
(43) Pub. Date: Aug. 9, 2018

(54) GRAPHITE BASED CHLORINE SENSOR

(71) Applicants: Si PAN, Burlington (CA); Raja GHOSH, Dundas (CA); M. Jamal DEEN, Dundas (CA)

(72) Inventors: Si PAN, Burlington (CA); Raja GHOSH, Dundas (CA); M. Jamal DEEN, Dundas (CA)

(21) Appl. No.: 15/749,232

(22) PCT Filed: Aug. 4, 2016
(86) PCT No.: PCT/CA2016/050914

§ 371 Date: Jan. 31, 2018

Related U.S. Application Data
(60) Provisional application No. 62/200,736, filed on Aug. 4, 2015.

(51) Int. Cl. G01N 27/30; G01N 33/18; G01N 27/416

(52) U.S. Cl. CPC G01N 27/301; G01N 27/4168; G01N 33/18

(57) ABSTRACT

Systems, methods, and devices relating to measuring free chlorine in samples. A graphite based electrode or sensor is provided. In conjunction with a reference electrode, a counter electrode, and a potentiostat, the electrode can be used to detect 2-6 ppm concentrations of free chlorine in liquid samples. The electrode can be manufactured from graphite used in pencil leads by electrochemical modification, with the graphite as the working electrode, and a suitable reference electrode, using an ammonium carbamate based electrolyte.

### TECHNICAL FIELD

The present invention relates to sensors. More specifically, the present invention relates to a graphite based sensor for use in sensing free chlorine in liquid samples.

### BACKGROUND

Chlorine is widely used as a disinfectant in the water treatment industry for inactivation of pathogenic microorganisms such as Cryptosporidium and Escherichia coli. Before chlorine treated water can be sent from the treatment plant into the distribution system, it must meet certain standards for residual free chlorine concentration, which is typically below the 5 ppm range. Free chlorine content in municipal water is currently measured using N,N′-diethyl-p-phenylenediamine (DPD) based colorimetry. There have been some efforts towards developing alternative detection methods, and improving or miniaturizing existing devices and methods. With increasing public awareness on water quality and tighter public health regulations and practices, such as point-of-use sampling and analysis, a robust, reliable, low-cost, and portable free chlorine sensor would be highly desirable. This is particularly relevant in small and remote communities, where highly-trained personnel may not be available, and routine maintenance is less feasible.

Several promising materials for free chlorine sensing with linear response have recently been reported in the literature. However, the sensing materials are either expensive (e.g. glassy carbon, gold, boron-doped diamond, graphene, carbon nanotubes, ferrocene), or potentially leach hazardous materials (e.g. benzethonium chloride, aniline oligomers). Moreover, in most cases, the upper range for sensing was 2.0 ppm, and hysteresis during repeated measurements was not systematically studied. In typical water-testing applications, the concentration of free chlorine in the tested sample is likely to fluctuate and hysteresis, if present, would affect sensor performance. Equally important is the selectivity of the sensor, i.e. ability to distinguish free chlorine from total chlorine, the latter being the combination of free chlorine and reduced chlorine in the form of chloride ions.

From the above, it is evident that there is a need for a free chlorine sensor that avoids the shortcomings of the prior art while addressing the needs of ease of use and suitability for rough, non-laboratory conditions.

### SUMMARY

The present invention provides systems, methods, and devices relating to measuring free chlorine in samples. A graphite based electrode or sensor is provided. In conjunction with a reference electrode and a potentiostate, the electrode or sensor can be used to detect 0-20 ppm concentrations of free chlorine in liquid samples. The electrode or sensor can be manufactured from graphite used in pencil leads by electrochemical modification, with the graphite as the working electrode, and a suitable reference electrode, using an ammonium carbamate based electrolyte.

In a first aspect, the present invention provides an electrode comprising:
at least one section comprising modified graphite;
   wherein
said electrode is for use in measuring a level of free chlorine in a liquid sample;
said modified graphite is modified by a process comprising:
   immersing graphite in an electrolyte solution with said graphite operating as a working electrode; and
   applying a voltage to said graphite such that there is a 1.0 V voltage potential difference between said working electrode and a reference electrode;
      wherein
said electrolyte comprises ammonium carbamate prepared in a sodium phosphate buffer.

In a second aspect, the present invention provides a process for modifying graphite, the process comprising:
immersing said graphite in an electrolyte solution with said graphite operating as a working electrode; and
applying a voltage to said graphite such that there is a 1.0 V voltage potential difference between said working electrode and a reference electrode;
   wherein a resulting modified graphite is used in an electrode for measuring free chlorine in a liquid sample.

### BRIEF DESCRIPTION OF THE DRAWINGS

The embodiments of the present invention will now be described by reference to the following figures, in which identical reference numerals in different figures indicate identical elements and in which:

FIG. 1 is an illustration of an experimental setup for modifying graphite according to one aspect of the invention;

FIG. 2 is a current-time profile obtained during the electrochemical modification of the graphite;

FIG. 3 illustrates the chronoamperometric response to increasing free chlorine concentration in the experimental setup shown in FIG. 1;

FIG. 4 is a graph showing the change in current in response to addition or removal of free chlorine from a sample being tested using the modified graphite electrode according to one aspect of the invention;

FIG. 5 is a block diagram of a system for testing for free chlorine according to another aspect of the invention;

FIG. 5A is a circuit diagram of an alternative setup for use in detecting chlorine according to one aspect of the invention; and

FIG. 5B is a circuit diagram of yet another alternative setup for detecting chlorine according to yet another aspect of the invention.

### DETAILED DESCRIPTION

As noted above, there is a need for a free chlorine measurement system that is cheap, easy to use, and is applicable to non-laboratory conditions. Many free chlorine sensors utilize the reaction between the sensor and amine groups on planar macrocyclic molecules. Building on this, and the fact that graphite is conductive due to its delocalized: bonds parallel to the crystal planes, the inventors considered that amine-modified graphite would be suitable for sensing free chlorine. More specifically, the 2p electron lone pair in an amine group would interact with the graphite through a p-n conjugation. Amine-modification of glassy carbon for sensing applications has been reported. As both graphite and glassy carbon have delocalized bonds, the same approach could be used with graphite.

One embodiment of the present invention employs ammonium carbamate to electrochemically modify common graphite to fabricate a graphite-based electrode for sensing free chlorine in water samples. This material is highly suitable for use in chronoamperometry. The proper functioning of this modified graphite does not require a periodically changed membrane. Also, the sensitivity of this modified material is high enough to effectively determine the free chlorine concentration in a relevant range (e.g. 0.1-2 ppm for municipal drinking water, higher ppm readings for vegetable and fruit washing processes).

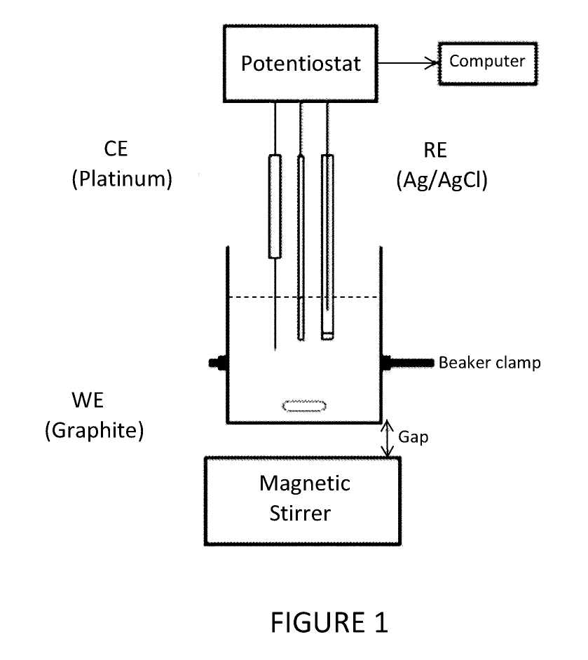

FIG. 1 shows an experimental setup according to one aspect of the invention. EmStat2 (manufactured by PalmSens BV, Utrecht, The Netherlands) was configured for the three-electrode chronoamperometry mode, for both electrochemical modification, and for carrying out the sensing experiments. All three electrodes were clamped fixed in position. A 10 mL beaker was separately clamped as shown in the figure to prevent direct contact with the magnetic stirrer, and thereby reduce interference in analysis. The stirring speed was maintained at a fixed rate of approximately 600 rpm. Liquid could be added to or removed from the beaker during the experiment without disturbing the electrodes. Evaporation loss from the beaker was not compensated for.

Pencil lead was cleaned using lab tissue and rinsed with deionized water. The electrochemical modification of the graphite surface was carried out at 1.0 V versus Ag/AgCl reference electrode using an electrolyte solution consisting of 0.1 M ammonium carbamate (292834-25 G) prepared in 0.1 M sodium phosphate buffer (pH 7.0), by mixing the two until the pH is 8.9. An auxiliary (or counter) platinum electrode was also used as the third electrode. In one experiment, the voltage (a potential of 1.0 V between the graphite working electrode and the reference electrode) was applied for approximately 7200 seconds. Regarding the temperature of the set-up, experiments have shown that a room temperature of between 19-21 degrees C. is preferred.

Free chlorine was sensed by chronoamperometry at 0.1 V versus Ag/AgCl reference electrode using the above described setup. The experiments were started with 10 mL of 100 mM sodium phosphate buffer (pH 7.0) in the beaker. Different volumes of sodium hypochlorite (425044-250 ML) stock solution were added to the beaker to simulate an increase in free chlorine concentration. Decrease in free chlorine was simulated by removing 1 mL of liquid from the beaker and replacing it with 1 mL of 100 mM sodium phosphate buffer (pH 7.0). The free chlorine concentration in the sodium hypochlorite stock solution was quantified by iodometry using sodium thiosulfate (SX0815-1, EMD, Mississauga, ON, Canada), potassium iodide (74210-140, Anachemia, Montreal, QC, Canada) and starch. The response to reduced chlorine was tested using 0.5 M NaCl (S7653-1KG) stock solution.

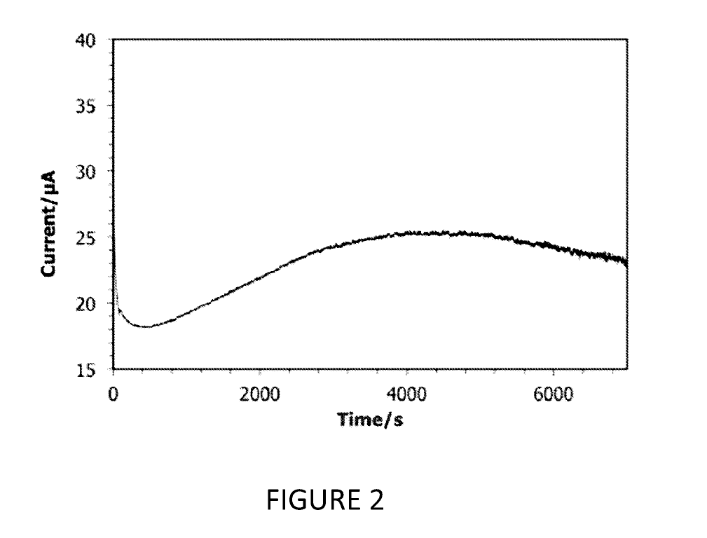

FIG. 2 shows the current-time profile obtained during electrochemical modification of the graphite surface. The current decreased first due to the depletion of carbamate close to the working electrode surface, then increased due to the activation of the working electrode surface, and finally decreased due to the decrease of available active site on the working electrode. The modification could be carried out using a simple setup and did not involve any harsh reaction conditions.

In one experiment, the modification was executed using only the graphite working electrode and a counter/reference electrode in conjunction with a subsystem for applying a voltage potential between the working electrode and the reference electrode such as a potentiostat. As in the above setup, the two electrodes were immersed in an ammonium carbamate-based electrolyte and a 1.0 V potential between the two electrodes was applied. As in the above experiment, the solution was a mixture of 0.1 M pH 7.0 sodium phosphate buffer and 0.1 M ammonium carbamate solution with a final pH of 8.9. Other suitable reference/counter electrodes may be used in the electrochemical modification, such as silver/silver chloride reference electrode, copper/copper sulphate reference electrode, saturated calomel reference electrode, etc. Other reference electrodes which do not have a passivation layer or a high-impedance salt bridge can be used in the electrochemical modification set-up.

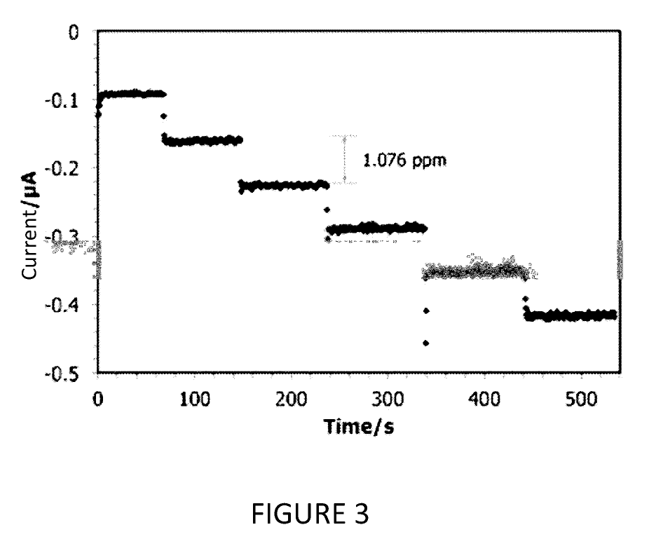

FIG. 3 shows the chronoamperometric response to increasing free chlorine concentration by 1.076 ppm per step. The decrease in current in each of the steps was comparable and the net decrease in current correlated linearly with quantity of free chlorine added. The sensitivity to free chlorine was 0.303 uA ppm−1 cm−2. The response was repeatable and insensitive to change of electrode area. The response time for 90% change (t90%) in signal was less than three seconds. The voltage of chronoamperometry was well outside the voltage range for dissolved oxygen. Therefore sample de-aeration was not required. The noise levels in these experiments (maximum fluctuation equivalent to 0.13 ppm) were lower than the reported values (0.69 and 1.33 ppm respectively) in the literature. It should be clear that, for the experiments carried out for FIG. 3, the voltage between the electrodes was kept constant at 0.1 V as the chlorine concentration was increased.

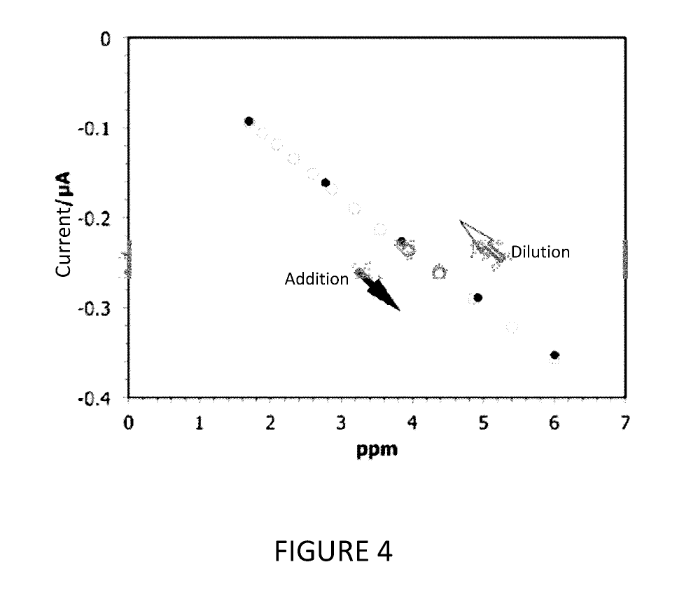

FIG. 4 shows the change in current in response to addition or removal of free chlorine from sample being tested. These results indicate very low hysteresis with the maximum hysteresis throughout the tested concentration range being 0.04 ppm at 6 ppm. In contrast to the literature without hysteresis study, these results indicate the real utility of repeatable readings in cases where free chlorine may increase or decrease.

As can be seen from FIG. 4, a correlation can be made between the current measured between the working electrode (made from the modified graphite) and the counter or auxiliary electrode and the free chlorine in the sample solution. A free chlorine measuring device with a three electrode configuration (a working electrode with modified graphite prepared as above, a counter or auxiliary electrode and a reference electrode), a potentiostat for applying and maintaining a voltage between the working electrode and the reference electrode, and a current measuring subsystem (which can measure current in the microampere range) can therefore be used to measure the free chlorine in a liquid sample. As with the experiments for FIG. 3, the voltage applied between the two electrodes was kept constant at 0.1 V.

In other experiments, several additions of 1.8 ppm NaCl were added to a solution, initially containing ˜2 ppm free chlorine. Results from these experiments show that the sensing technique was highly selective towards free chlorine and the addition of chloride ions elicited no response. This ability to distinguish free chlorine from chloride ions is useful in sensors for water applications as municipal water contains variable quantities of chloride ions.

It is desirable that a sensor be suitable for repeated use in a highly reproducible manner. One of the graphite electrodes used in the experiments described above was stored in deionized water for a period of several months without any deterioration in performance during repeated use following storage.

Most chemicals used in the experiments were purchased from Sigma-Aldrich and used as received. Those obtained from other suppliers are specifically identified. The Ag/AgCl reference electrode (CHI111) and the platinum wire counter electrode (CHI115) were purchased from CH Instruments, Inc. (Austin, Tex.). The reference electrode was filled with 1 M KCl (P217-500, Fisher Scientific, ON, Canada) solution. The pencil lead (TrueColor, 2B, 0.7×100 mm) was purchased from TrueColor Co., Ltd (Kunshan, Jiangsu, China). The pencil lead used in the experiments was rated as a 2B pencil in terms of hardness and darkness. Other pencil leads with different ratings for hardness and darkness may, of course, be used. The pencil lead composition was determined to be a mixture of graphite and clay. Other compositions may be used as long as the majority of the composition is graphite.

It should be noted that the modified graphite based electrode was also tested against regular municipal water samples and the free chlorine concentrations were verified using DPD colorimetry.

It should also be noted that the graphite-based electrode or sensor can have any number of configurations. Specifically, while FIG. 1 contemplates a rod-like configuration for the electrode, other configurations are possible. As an example, the electrode may be a sheet of modified graphite, a planar sensor, a portion of a larger electrode with the rest of the electrode being configured to conduct electricity, or it may even be deposited on to a suitable substrate (e.g. pencil marks on paper). In addition to the above, the graphite-based electrode or sensor may be packaged together with suitable reference and/or reference/counter electrodes for use together in a chlorine measuring device.

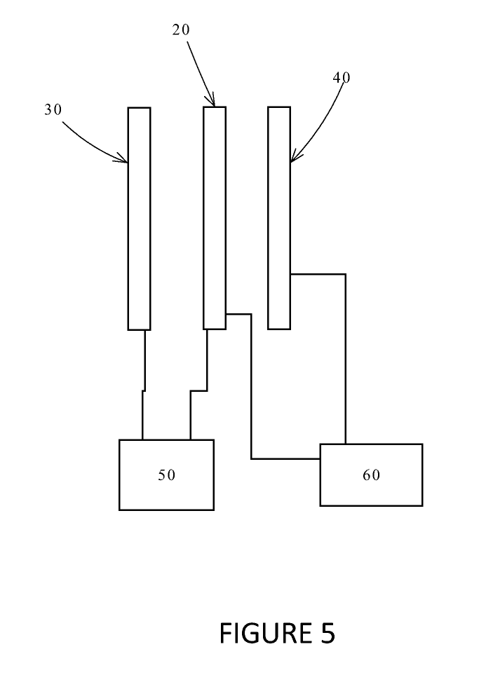

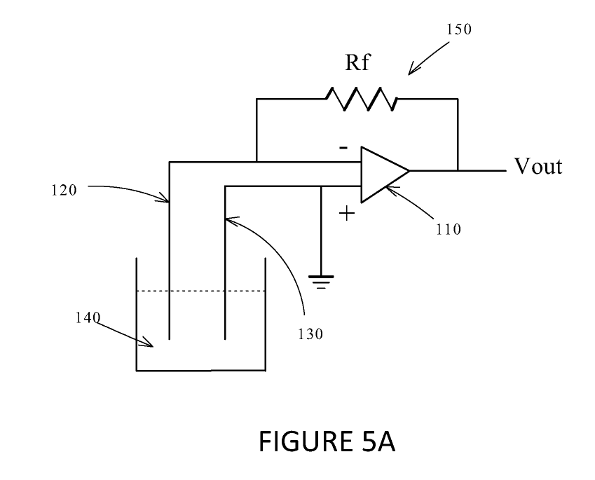

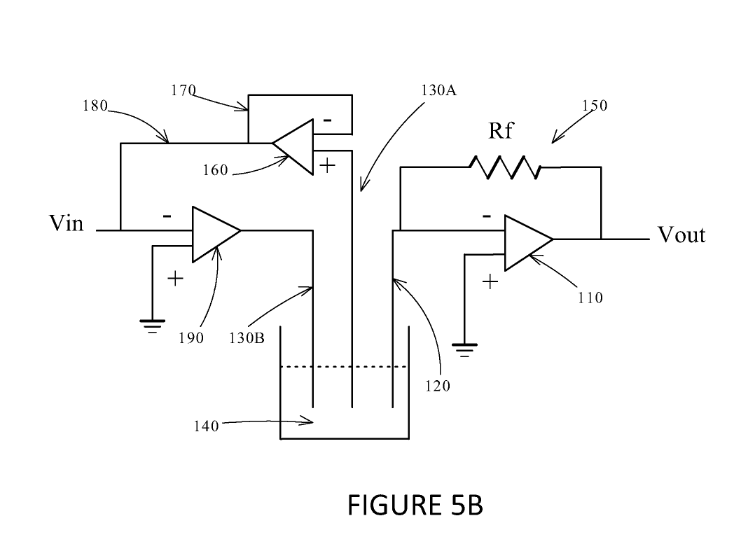

Referring to FIG. 5, a block schematic diagram of a system according to one aspect of the invention is illustrated. The system 10 has a working electrode 20, a reference electrode 30, and a counter electrode 40. These three electrodes would be immersed in the sample to be tested. These electrodes are coupled to a potentiostat 50 that provides a voltage potential between the working electrode and the reference electrode. An ammeter 60 would measure a current between the working electrode and the counter electrode. The ammeter reading would determine the free chlorine concentration in the liquid sample. Suitable circuitry can, of course, be used to calibrate an output reading from the ammeter which is more user friendly and easier to understand to the layperson.

Referring to FIG. 5A, an alternative circuit to the system in FIG. 5 is illustrated. This other aspect of the invention is a system 100 that uses an operational amplifier 110, a working electrode 120, and a reference/counter electrode 130. The working electrode 120 and the reference/counter electrode 130 are immersed in the sample 140 to be tested. For the operational amplifier 110, the negative input is coupled to the amplifier output by way of a resistance 150. This negative input is also coupled to the working electrode 120. The positive input of the operational amplifier 110 is also coupled to the reference/counter electrode 130 as well as to ground.

Another alternative to the system in FIG. 5 is illustrated in FIG. 5B. In this alternative, the operational amplifier 110 has its output still coupled to the working electrode 120 and to the negative input by way of a resistance 150. The positive input is coupled to ground. The reference electrode 130A is coupled to the positive input of a second operational amplifier 160. The negative input 170 of this second operational amplifier 160 is looped to connect to the output 180 of operational amplifier 160. This output of operational amplifier 160 is coupled to an input voltage Vin and to the negative input of a third operational amplifier 190. The output of this third operational amplifier 190 is coupled to the counter electrode 130B which is also immersed in the sample 140 along with the reference electrode 130A and the working electrode 120. The positive input of operational amplifier 190 is coupled to ground.

For the alternative setups in FIGS. 5A and 5B, the working electrode is constructed using modified graphite as explained above.

In addition to its use for detecting free chlorine, the modified graphite-based electrode can also be used for detecting and measuring combined chlorine. As is well-known, combined chlorine is the free chlorine that has reacted with ammonia or organic amines to form chloramines, namely monochloramine, dichloramine, trichloramine and other organic chloramines. The sum of combined chlorine and free chlorine are referred to as total chlorine. While the combined chlorines have weaker effects inhibiting the microorganisms, they are still reactive and are in equilibrium with free chlorine.

Chloramines can be detected using the setups detailed above by setting a different voltage on the working electrode. Chloramines can also be detected by studying the kinetics from current-time curves to deconvolute free chlorine and chloramine(s).

The performance parameters of the modified graphite sensing material includes linear, fast, and low-noise response with low hysteresis to free chlorine concentrations while having no response to the reduced form—chloride ions that have no disinfection potency unlike free chlorine. The short response time of this material also allows it to be used in-line and provide real-time monitoring data.

In addition to good performance features, the lower cost and ease-of-use are two useful characteristics for a free chlorine sensor in less developed countries where water quality is more of a desired property without complex infrastructure or specifically trained personnel. The graphite based sensor also does not put a heavy impact on the environment, both in its manufacturing and actual use—the fabrication is benign chemistry and no hazardous chemicals leach to the water being tested. The electrochemical fabrication can be easily scaled up for mass production, chemically and economically. Hand-drawn sensors are also possible because the material is based on pencil lead. (A few instances have been developed and relevant responses have been obtained by the inventors.) The sensor material offers possibilities of integration with electronics and software for autonomous and personnel-free sensors.

This sensor material has been stored in water for several months without loss of sensing capability. The unusual features include the use of the widely available pencil lead as the base of the sensing material. Compared to some other devices, a device using this material does not require repeated change of hydrophobic membrane cap on the sensing probe. No chemical precursors are required to react with free chlorine, e.g. generating colours to measure, etc.

A sensor using this material is ideal for rural area and distant communities. It can be deployed to such regions or put in grocery stores along with other everyday supplies. Integrated sensors using this material can be easy to use and require little maintenance.

In addition to the above advantages, the material has shown low hysteresis. The signal had identical values when the concentrations of free chlorine were identical, regardless of an increasing or decreasing change of free chlorine concentrations prior to the measurement of free chlorine concentration. Other previous reports on free chlorine-sensing materials included no such tests and lacked proof-of-principle for real-world applications.

The noise level of this material, when operated in chronoamperometry, has proven superior to existing or proposed materials. The response time of this graphite based material is less than four seconds under tested conditions. The material has shown to be stable for at least seven weeks in storage in water without special care, and the operation of the free chlorine sensor does not require any specialist knowledge.

A person understanding this invention may now conceive of alternative structures and embodiments or variations of the above all of which are intended to fall within the scope of the invention as defined in the claims that follow.

We claim:

1. An electrode comprising:
at least one section comprising modified graphite;
wherein
said electrode is for use in measuring a level of chlorine in a liquid sample;
said modified graphite is modified by a process comprising:
immersing graphite in an electrolyte solution with said graphite operating as a working electrode; and
applying a voltage to said graphite such that there is a 1.0 V voltage potential difference between said working electrode and a reference electrode;
wherein
said electrolyte comprises ammonium carbamate prepared in a sodium phosphate buffer.

2. An electrode according to claim 1, wherein said ammonium carbamate has a concentration of 0.1 M.

3. An electrode according to claim 1, wherein said sodium phosphate buffer has a concentration of 0.1 M and a pH of 7.0.

4. An electrode according to claim 1, wherein said reference electrode is an Ag/AgCl reference electrode.

5. An electrode according to claim 1, wherein said voltage is applied for at least approximately 3600 seconds.

6. An electrode according to claim 1, wherein said process includes using an auxiliary electrode.

7. An electrode according to claim 6, wherein said auxiliary electrode is a platinum electrode.

8. An electrode according to claim 1, wherein said electrolyte solution has a pH of 8.9 prior to an application of said voltage.

9. An electrode according to claim 5, wherein said voltage is applied for at least approximately 4800 seconds.

10. An electrode according to claim 5, wherein said voltage is applied for at least approximately 7200 seconds.

11. A process for modifying graphite, the process comprising:
immersing said graphite in an electrolyte solution with said graphite operating as a working electrode; and
applying a voltage to said graphite such that there is a 1.0 V voltage potential difference between said working electrode and a reference electrode;
wherein a resulting modified graphite is used in an electrode for measuring chlorine in a liquid sample.

12. A process according to claim 11, wherein said ammonium carbamate has a concentration of 0.1 M.

13. A process according to claim 11, wherein said sodium phosphate buffer has a concentration of 0.1 M and a pH of 7.0.

14. A process according to claim 11, wherein said reference electrode is an Ag/AgCl reference electrode.

15. A process according to claim 11, wherein said voltage is applied for at least approximately 3600 seconds.

16. A process according to claim 11, wherein said process includes using an auxiliary electrode.

17. A process according to claim 16, wherein said auxiliary electrode is a platinum electrode.

18. A process according to claim 15, wherein said voltage is applied for at least 4800 seconds.

19. A process according to claim 15, wherein said voltage is applied for at least 7200 seconds.

20. A process according to claim 11, wherein said electrolyte solution has a pH of 8.9 prior to an application of said voltage.
